(19) United States
(12) Patent Application Publication
(10) Pub. No.: US 2006/0276777 A1
(43) Pub. Date: Dec. 7, 2006

(54) Corneal shaping

(76) Inventor: Minas Theodore Coroneo, Vaucluse (AU)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 11/400,061

(22) Filed: Apr. 7, 2006

Related U.S. Application Data
(60) Provisional application No. 60/669,633, filed on Apr. 8, 2005.

(51) Int. Cl. A61B 18/18

(52) U.S. Cl. 606/5

(57) ABSTRACT

Compositions and methods for moulding corneal tissue in a patient for correcting or improving refractive errors in the eye, and in particular, compositions and methods for sequentially softening then hardening the corneal tissue, preferably by manipulation of matrix metalloproteinase activity in the cornea.

### FIELD OF THE INVENTION

The present invention relates to methods for reshaping of the cornea in an eye of a patient, and in particular, to the use of corneal softening compounds that enhance corneal reshaping methods, particularly for treating refractive errors of the eye.

### BACKGROUND OF THE INVENTION

The cornea is a transparent, dome-shaped region that covers the front of the eye. It provides a powerful refracting surface of ⅔ of the eye's focusing power.

The adult cornea is approximately ½-1 millimetre in thickness, and consists of 5 layers: the corneal epithelium, Bowman's membrane, the corneal stroma, Descemet's membrane and the endothelium.

The epithelium is about 5-6 cell-layers thick, and overlies Bowman's membrane, which is a very tough layer that is difficult to penetrate, and protects the cornea from injury.

The stroma is the thickest layer and lies just beneath Bowman's membrane. It consists of tiny collagen fibrils that run parallel to each other.

Descemet's membrane lies between the stroma and the corneal endothelium, the latter layer being only one cell-layer thick.

A misshapen cornea can contribute to refractive errors of the eye: when the axial length of the eye is too short, relative to the focusing power, the result is hyperopia (farsightedness), and when it is too long, the result it myopia (nearsightedness).

Optical devices such as glasses and contact lenses have been used to treat the above-mentioned refractive errors, where both types of devices working by changing the angle at which light enters the cornea via refraction of incoming light. Despite the success of such devices in treating refractive errors of the eye, many patients requiring such devices find that their use is inconvenient and at times, uncomfortable.

In modern refractive surgery, a principal approach is to alter the shape of the cornea. This is generally achieved by surgical procedures such as LASIK (laser in situ keratomilieusis) where a flap is cut in the cornea and tissue ablated using an excimer laser. In variants of this procedure, such as LASEK (laser epithelial keratomileusis) or PRK (photorefractive keratectomy), the flap is superficial or the surface epithelium is removed and subsequently regenerates but corneal tissue is still removed to effect a change in corneal shape. Corneal flap creation and tissue removal both may weaken the cornea, resulting in ectasia, an anterior bulging of cornea which creates an irregularity in corneal shape, resulting in astigmastism that cannot easily be corrected by spectacle or contact lenses and may require a corneal graft to restore sight. These procedures are destructive of corneal tissue but in the medium term are enjoying success and popularity. Ectasia may be a long term complication of these procedures. Furthermore, these procedures may not achieve ideal shaping of the cornea, which means the patient is still reliant on contact lenses or glasses to a certain degree.

In general, reconstructive procedures of the eye, in which tissue is not sacrificed, offer advantages. In orthokeratology, which is used particularly to treat myopia (short-sightedness), hard contact lenses are used to mould and therefore reshape the cornea. The mold is a specially designed contact lens, somewhat larger than a standard lens but of similar appearance. The lens flattens the cornea as it is worn, which may be during sleep. The procedure takes hours (in mild cases) to months (in difficult cases) to reach good functional vision. The contact lens wearing time is then gradually reduced until a minimal-wear time is established that maintains the corneal shape and good functional vision. Corneal shape can be fine-tuned by minor modifications to the retainer mold. Rigid Gas Permeable contact lenses for overnight treatment have been given FDA approval. Disadvantages of this technique include reliance on contact lenses (even if minimal) and thus a “non-permanent” cure and contact lens related infections (which can be sight threatening). The lack of persistence of altered corneal shape has been taken to indicate that the cornea is either highly elastic or has some other memory mechanism. The advantage of this procedure is that no corneal tissue is removed, and thus the corneal integrity is not compromised and there is no surgery.

It is evident from the methods currently in practice that there remains a need for improved methods for corneal shaping, in the context of both surgical and non-surgical corneal re-shaping technologies.

### SUMMARY OF THE INVENTION

The present inventor has found that manipulation of certain enzymes within the cornea result in alterations in the softness of the corneal tissue.

Accordingly, in a first broad aspect, the present invention relates to a method of moulding corneal tissue in an eye of a patient, the method comprising:
administering to the patient an effective amount of one or more compounds that activate at least one matrix metalloproteinase enzyme in the cornea and thereby soften the cornea;
providing a shaping means to the patient's cornea while said cornea is in a softened state until a desired corneal shape has been achieved;
ceasing the use of said shaping means.

Preferably, the shaping means is a contact lens.

Even more preferably, the one or more compounds that activate at least one matrix metalloproteinases enzyme in the cornea are selected from fluoroquinolone antibiotics, non-steroidal anti-inflammatory drugs (NSAIDS) and prostaglandin F(2alpha)(PGF (2alpha)) analogues.

In a particularly preferred form, the fluoroquinolone antibiotic is selected from ciprofloxacin, clinafloxacin, enoxacin, fleroxacin, gatifloxacin, gemifloxacin, grepafloxacin, levofloxacin, lomefloxacin, moxifloxacin, nalidixic acid, nomofloxacin, norfloxacin, ofloxacin, pefloxacin, sitafloxacin, sparfloxacin, temafloxacin and trovafloxacin, or any combination thereof.

In an even more preferred form, the one or more compounds that activate at least one matrix metalloproteinase in the cornea are a fluoroquinolone antibiotic and a non-steroidal anti-inflammatory agent.

Preferably, the desired corneal shape is retained by administration of an effective amount of one or more compounds that return the cornea to a hardened stated following administration of the one or more compounds that activate at least one matrix metalloproteinase enzyme in the cornea.

In a preferred form, the one or more compounds that return the cornea to a hardened state are selected from inhibitors of one or more matrix metalloproteinases in the cornea, doxycycline, triptolide, cross-linking agents such as aldehydes, oxidative hardening agents such as copper sulfate and iron sulfate, enzymes such a lysyl oxidase and enzymes that hydroxylate collagen residues, enzymes that otherwise induce protein modifications that enhance corneal rigidity, corticosteroids, curcuminoids, galardin and medroxyprogesterone.

In a particularly preferred form, the matrix metalloproteinase inhibitor is selected from doxycycline, triptolide, corticosteroids and curcuminoids

Preferably, the one or more compounds that activate one or more matrix metalloproteinases in the cornea are administered orally.

In another preferred form, the one or more compounds that activate one or more matrix metalloproteinases in the cornea are administered topically.

In a further preferred form, the one or more compounds that activate one or more matrix metalloproteinases in the cornea are administered via a contact lens.

In yet another preferred form, the one or more compounds that activate one or more matrix metalloproteinases in the cornea are administered intra-ocularly.

In still another preferred form, the one or more compounds that activate one or more matrix metalloproteinases in the cornea are administered parenterally.

In a further preferred form, the one or more compounds that return the cornea to a hardened state are administered orally.

Preferably, the one or more compounds that return the cornea to a hardened state are administered topically.

In a further preferred form, the one or more compounds that return the cornea to a hardened state are administered via a contact lens.

In yet another preferred form, the one or more compounds that return the cornea to a hardened state are administered intra-ocularly.

In still another preferred form, the one or more compounds that return the cornea to a hardened state are administered parenterally.

Preferably, the moulding of the cornea is part of an orthokeratology procedure.

In a further preferred form, the moulding of the cornea is performed to enhance an outcome of a LASIK procedure.

In still another preferred form, the moulding of the cornea is performed to enhance an outcome of a LASEK or PRK procedure.

In yet another preferred form, the moulding of the cornea is performed following cataract surgery, wherein a replacement lens that is used in said cataract surgery that does not provide sufficient visual acuity.

In another broad form, the present invention relates to a composition for softening corneal tissue in a patient, said composition comprising an effective amount of one or more compounds that activate at least one matrix metalloproteinase enzyme in the cornea, optionally in association with one or more pharmaceutically acceptable carriers or excipients.

Preferably, the one or more compounds that activate at least one matrix metalloproteinases enzyme in the cornea are selected from fluoroquinolone antibiotics, non-steroidal anti-inflammatory drugs (NSAIDS) and prostaglandin F(2alpha)(PGF (2alpha)) analogues.

Even more preferably, the fluoroquinolone antibiotic is selected from ciprofloxacin, clinafloxacin, enoxacin, fleroxacin, gatifloxacin, gemifloxacin, grepafloxacin, levofloxacin, lomefloxacin, moxifloxacin, nalidixic acid, nomofloxacin, norfloxacin, ofloxacin, pefloxacin, sitafloxacin, sparfloxacin, temafloxacin and trovafloxacin, or any combination thereof.

In a particularly preferred form, the one or more compounds that activate at least one matrix metalloproteinase are a fluoroquinolone antibiotic and a non-steroidal anti-inflammatory agent.

In a further broad form, the present invention relates to a composition for hardening corneal tissue in a patient following corneal softening by activation of one or more matrix metalloproteinases, said composition comprising one or more corneal-hardening agents, optionally in association with one or more pharmaceutically acceptable carriers or excipients.

Preferably, the one or more corneal-hardening agents are selected from an inhibitor of one or more matrix metalloproteinases in the cornea, doxycycline, triptolide, cross-linking agents such as aldehydes, oxidative hardening agents such as copper sulfate and iron sulfate, enzymes such a lysyl oxidase and enzymes that hydroxylate collagen residues, enzymes that otherwise induce protein modifications that enhance corneal rigidity, corticosteroids, curcuminoids, galardin and medroxyprogesterone.

Even more preferably, the matrix metalloproteinase inhibitor is selected from doxycycline, triptolide, corticosteroids and curcuminoids

### DETAILED DESCRIPTION OF THE INVENTION

Throughout this specification, unless the context requires otherwise, the words “comprise,” “comprises” and “comprising” will be understood to imply the inclusion of a stated step or element or group of steps or elements but not the exclusion of any other step or element or group of steps or elements.

The reference to any prior art in this specification is not, and should not be taken as, an acknowledgement or any form of suggestion that the prior art forms part of the common general knowledge in Australia.

It has surprisingly been found by the present inventor that compounds which soften the cornea by activation of matrix metalloproteinase expression in the cornea, thereby softening the cornea, can enhance the efficacy of a corneal reshaping procedure in an eye of a patient.

Thus, in a first broad form the present invention relates to a method and compositions for moulding corneal tissue in an eye of a patient.

In a preferred form, the method comprises:
administering to the patient an effective amount of one or more compounds that activate at least one matrix metalloproteinase enzyme in the cornea and thereby soften the cornea;
providing a shaping means to the patient's cornea while said cornea is in a softened state until a desired corneal shape has been achieved;
ceasing the use of said shaping means.

The term “patient” refers to patients of human or other mammal and includes any individual it is desired to examine or treat using the methods of the invention. However, it will be understood that “patient” does not imply that symptoms are present. Suitable mammals that fall within the scope of the invention include, but are not restricted to, primates, livestock animals (e.g. sheep, cows, horses, donkeys, pigs), laboratory test animals (e.g. rabbits, mice, rats, guinea pigs, hamsters), companion animals (e.g. cats, dogs) and captive wild animals (e.g. foxes, deer, dingoes).

By “effective amount,” in the context of treating or preventing a condition is meant the administration of that amount of active to an individual in need of such treatment or prophylaxis, either in a single dose or as part of a series, that is effective for treatment of, or prophylaxis against, that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

The term “activates”, as used herein, encompasses any mode or means of increasing the activity of at least one matrix metalloproteinase (MMP) enzyme in the cornea, such as, but not limited to, allosteric activation of an MMP and increases in the level of expression of the MMP for example, by increased transcription and/or translation of the MMP.

Certain compounds are known to activate matrix metalloproteinase in the cornea, which is considered an undesirable effect as it can lead to structural changes in the eye, such as epithelial adhesive abnormalities (Saghizadeh et al., Am J Pathol. 2001 February; 158(2):723-34) and keratoconus (Collier, Clin Experiment Ophthalmol. 2001 December; 29(6):340-4). These compounds include, but are not limited to, fluoroquinolone antibiotics, non-steroidal anti-inflammatory drugs (NSAIDS) (Reviglio et al. BMC Opthalmol. 2003; 3:10), prostaglandin F(2alpha)(PGF (2alpha)) analogues (Viestenz et al., Klin Monatsbl Augenheilkd 2004 September; 221 (9):753-6), or any combination thereof.

Fluoroquinolone antibiotics are a group of broad spectrum antibiotics that target the bacterial enzyme DNA gyrase. It has been reported that a side effect of treatment with these drugs is Achilles tendon pain and/or rupture (Pierfitte C, Royer R J.—Tendon disorders with fluoroquinolones. Therapie. 1996; 51:419-20.). The effects of topical ciprofloxacin, ofloxacin and levofloxacin on MMP levels in rat cornea have recently been reported (Reviglio et al. BMC Opthalmol. 2003; 3:10). MMP's 1, 2, 8 and 9 were found to be increased by these medications in the corneal epithelium and anterior stroma both in animals with epithelial defects but significantly also in those with intact epithelium.

The present inventor has recently established that the fourth generation fluoroquinolone, gatifloxacin is also capable of activating matrix metalloproteinases.

Accordingly, suitable fluoroquinolone antibiotics include, but are not limited to, ciprofloxacin, clinafloxacin, enoxacin, fleroxacin, gatifloxacin, gemifloxacin, grepafloxacin, levofloxacin, lomefloxacin, moxifloxacin, nalidixic acid, nomofloxacin, norfloxacin, ofloxacin, pefloxacin, sitafloxacin, sparfloxacin, temafloxacin, trovafloxacin and medroxyprogesterone or any combination thereof.

Nonsteroidal anti-inflammatory drugs (NSAIDS) are used commonly on topical agents in eye conditions (diclofenac, ketorolac tromethamine and flurbiprofen). Diclofenac and ketorolac induce expression of MMPs 1, 2 and 8 in rat corneas (Reviglio et al., above).

In a particularly preferred embodiment, the MMP-activating composition is a combination of one or more fluoroquinolone antibiotic and one or more NSAIDs. This combination of MMP activators not only provides a dual mechanism of MMP expression, but also offers the advantage of the therapeutic protection of an antibiotic and an anti-inflammatory, which reduces the risks of ocular infection and the dangers of activation of the inflammatory cascade in the ocular environment.

In a further embodiment, softening and moulding of the cornea is followed by administration of an effective amount of a corneal hardening agent, which assists in retaining the cornea in a desired, re-moulded condition.

Preferably, the compounds for hardening the cornea include, but are not limited to inhibitors of matrix metalloproteinases activity such as, but not limited to, doxycycline, triptolide (Lu et al. Invest Ophthalmol Vis Sci. 2003 December; 44(12):5082-8), corticosteroids (Lu et al., Invest Ophthalmol Vis Sci 2004 September; 45(9):2998-3004), curcuminoids (Mohan et al., J Biol Chem 2000 Apr. 7; 275(14):10405-12), and galardin (Hao et al., Exp Eye Res. 1999 May; 68(5):565-72).

Other suitable compounds for hardening the cornea include, but are not limited to, cross-linking agents such as aldehydes, oxidative hardening agents such as copper sulfate and iron sulfate, and enzymes such a lysyl oxidase and enzymes that hydroxylate collagen residues, and enzymes that otherwise induce protein modifications that enhance corneal rigidity.

One chemical reaction in which aldehydes frequently engage is called the aldol condensation reaction. In one aspect of the present invention, aldehydes are reacted with each other to form cross links within corneal components using the aldol condensation reaction. In a typical aldol condensation reaction, the carbonyl group undergoes an enolization where an enolate anion is formed. An enolate anion is formed when one pair of electrons is shifted to the carbon of the carbonyl group from a neighboring carbon atom. A proton acceptor may remove a proton from the neighboring carbon atom in the reaction, and if that acceptor is a hydroxyl then water is formed. As the electrons shift to the carbon of the carbonyl group a double bond is formed between it and the neighboring carbon atom. This shift in electrons causes a pair of electrons to shift from the carbonyl carbon to the carbonyl oxygen, creating a negative charge on that oxygen. The resulting carbon-carbon double bond of the enolate reaction is extremely reactive.

The optimum concentration of glyceraldehyde may vary depending on the protocol, the nature of the delivery vehicle, and the number of administrations. In general, concentrations of glyceraldehyde will vary within the range of about 0.01% to 10% weight to volume (w/v). In one embodiment, the concentration range of the glyceraldehyde solution will vary from 1% to 5% (w/v). In still another embodiment, the concentration of 3% glyceraldehyde is used.

Aldehydes other than glyceraldehyde are also contemplated for use in the present invention. Such compounds include acetaldehyde, glyceraldehyde, phenylacetaldehyde, valeraldehyde, 3,4-dihydroxyphenylacetaldehyde, glycoaldehyde (the aldehyde form of ethylene glycol), pyruvaldehyde, dihydroxy acetone, acetol, glyoxal, and mutarotational isomers of aldehydes including glucose, fructose, lactose, etc. Suitable alternative aldehydes have biochemical characteristics similar to those of glyceraldehyde possessing .alpha.-hydrogen, including biodegradability, low toxicity, and ready readsorption into the treated area.

The corneal softening and hardening chemicals, such as various agents and enzymes, used in the methods and compositions of the present invention, in addition to the recommended dosages of such compounds and enzymes, can be determined by one of skill in the art through routine experimentation. Such experimentation can comprise testing a dose of an enzyme or agent on donor globes (eyes) mounted in perfusion chambers (Bahler et al., Am J Ophthalmol. 2004 December; 138(6):988-94) or testing such a dose on laboratory animals. Briefly, to determine an appropriate corneal hardening amount of a known softening compound or enzyme, or an agent or enzyme to be tested for its ability to produce corneal hardening, a dose of the agent or enzyme is administered to a cornea in a donated eye, the cornea of which can be organ cultured, and the softening and toxic effect of the agent is thereafter determined. Alternatively, a test animal may be used in order to establish appropriate dosages of corneal softening and hardening agents.

In order to determine whether an enzyme or agent is effective in softening a cornea without producing toxicity, or, if it is a known softening agent, whether a particular dosage will produce corneal hardening without causing toxicity, the enzyme or agent is first mixed in a carrier vehicle that is pharmaceutically acceptable to a mammal. Preferably, the enzyme or agent is in lyophilized (dry powder) form, and is dissolved in isotonic saline. However, one of ordinary skill in the art will understand that a variety of pharmacologically acceptable carriers which do not interfere with the functioning of an enzyme or agent can be used.

A test dose of the enzyme or agent in solution is then administered to a test cornea in order to determine its corneal softening and toxic effect. In one procedure for testing candidates, the test enzyme or agent is first administered to donor globes (eyes from a human donor) mounted in plastic sockets. This procedure is particularly preferred for determining the effect of an enzyme or agent on a human cornea because in this way a human cornea can be tested without subjecting a living person to experimentation. A donor globe used in this procedure is prepared for experimentation by injecting it with sufficient saline to maintain intraocular pressure of the globe at approximately 20 mm Hg.

The test dose of enzyme or agent is then administered to the donor cornea. Such administration can be, for example, by applying the drug topically as an eye drop or by injection of the enzyme into the cornea. Normally, the lens will become opacified following this step due to the introduction of water into the eye and a change in the refractive index of the eye. After a test period of time, the mounted globe is then examined to determine whether any corneal hardening or toxicity has occurred, and if so the extent of such hardening and toxicity.

The examination of the cornea can be performed, for example, through slit-lamp examination to determine the clarity of the cornea, or by in vivo confocal microscopy, pachymnetry to measure the thickness of the cornea; computer-assisted corneal topography to evaluate surface topographical changes; measurement of the tensile strength of the cornea; measurement of the distensibility of the cornea; keratometry to measure central corneal curvature; and retinoscopy to measure the refractive error of the cornea. The values determined from these tests are compared to values determined prior to the administration of the agent or enzyme.

In addition, a treated cornea in a mounted globe can be subjected to a number of other tests to determine the strength and viability of the cornea following treatment. For example, light, scanning, x-ray diffraction analysis, and transmission electron microscopy can be used to examine the morphology of the cornea; tissue culture is prepared to determine the viability of the cells of the cornea following treatment; biochemical studies can be made of the collagens and other structural components of the cornea following treatment.

The foregoing tests of donated globes and corneas can be used to verify that use of a particular enzyme or agent does not compromise the transparency of the cornea, decrease the viability of the corneal cells, or damage the structural integrity of the cornea. Testing the use of an enzyme or agent on the cornea of a test animal, however, is also desirable in order to make sure that the candidate has no unexpected effect in living mammals that is not discovered during tests of donated eyes. In order to test the effect of a particular test enzyme or agent, a test dose in a pharmacologically acceptable carrier solution is administered to a test animal, in this case a mammal, so as to deliver that agent to the cornea of the animal.

The corneal re-shaping procedure may be non-surgical, such as orthokeratology, or may follow surgical re-shaping of the eye after a procedure such as LASIK, LASEK or PRK, the latter two procedures involving removal of corneal tissue via surgical intervention. Alternatively or additionally, the corneal re-shaping procedure may be employed following cataract surgery, where the power of the lens that is used to replaced the cataractous lens in a patient does not provide sufficient visual acuity.

Compositions according to the invention may be formulated with standard buffers, excipients, carriers, diluents and the like. Examples of carriers include: water, physiologically saline, isotonic solutions containing dextrose, glycerol or other agents conferring isotonicity, lower alcohols, vegetable oils, polyethylene glycol, glycerol triacetate and other fatty acid glycerides. Examples of other carriers which may be used include cream forming agents, gel forming agents, and the like, compounding and tabletting agents. Excipients include buffers, stabilisers, emulsion forming agents, colouring compounds, salts, amino acids, antibiotics and other anti-bacterial compounds chelating agents and the like. More than one excipient and carrier may be used.

The foregoing enzymes and agents for softening or hardening a cornea may be administered in any way known to the art. For example, in one embodiment, an enzyme or agent is injected directly into the eye in a location proximal to the cornea.

In another embodiment of the present invention, corneal softening compounds are administered to the eye of a subject by topical application in the form of eye drops. A sufficient number of drops are applied so as to administer a desired concentration of enzyme or agent to the cornea of the subject. The eye drop method of administration may be superior to injection based administration based on the less discomfort to the cornea of the subject resulting from an injection technique.

In still another embodiment, alternative means of aiding diffusion across the eye into the cornea may be used. Such means include, for example, the use of liposomes to deliver the active enzyme or agent. The enzyme or agent is packaged into liposomes, which can pass across the lipid soluble membrane of the corneal epithelium and into the corneal stroma.

Other means of aiding diffusion include the use of an electrical current to make the outer membrane of the eye more permeable to the passage of enzymes and agents, known as iontophoresis. Using this procedure, an electrical current travelling through a salt solution causes the agents to pass into the eye as charged particles.

Compounds that enhance the ability of the active compounds of the present invention to penetrate the cornea are contemplated. A variety of compositions are envisioned for use as vehicles by which to administer the active agents of the present invention to the eye of a subject mammal. Suitable substances and means include acidifying agents, aerosol propellants, air displacement, alcohol denaturants, alkalizing agents, anticaking agents, antifoaming agents, antimicrobial preservatives, antioxidants, buffering agents, capsule lubricants, chelating agents, coating agents, colouring agents, complexing agents, desiccants, emulsifying and/or solubilizing agents, filtering aids, flavours and perfumes, glidants and/or anticaking agents, humectants, ointment bases, plasticizers, polymer membranes, solvents, sorbents, carbon dioxide, stiffening agents, suppository bases, suspending and/or viscosity-increasing agents, sweetening agents, tablet binders, tablet and/or capsule diluents, tablet disintegrants, tablet and/or capsule lubricants, tonicity agents, viscosity increasing, water repelling agents and wetting and/or solubilizing agents.

In alternative embodiments, sustained release vehicles are used. Sustained release vehicles are compositions that act to hold the active ingredients of the present invention in functional association with the cornea. Compounds and compositions in the sustained release technology are well known in the art. (See, Controlled Drug Delivery, 2.sup.nd ed., Joseph R. Robinson & Vincent H. L. Lee, Eds., Marcel Dekker, Inc., New York, 1987). By holding the active ingredients in association with the cornea to be treated, a sustained release vehicle acts to increase the efficacy of the active ingredients of the present invention. This increase in efficacy can be attributed to the sustained release vehicle acting to raise the local concentration of the active ingredients of the present invention with respect to the treated cornea to levels higher than would be possible without the sustained release vehicle.

Sustained release vehicles for use with the present invention hold or localise the active agents of the present invention in proximity to the cornea and have no detrimental effects on the cornea or the activity of the agents of the present invention. In a preferred embodiment, the sustained release vehicle is water soluble. Examples of suitable sustained release vehicles include: cellulose ethers such as methyl cellulose, methylhydroxypropyl cellulose, methylhydroxyethyl cellulose, hydroxypropyl cellulose, hydroxyethyl cellulose, and sodium carboxymethyl cellulose. Cellulose esters such as cellulose acetate phthalate and hydroxypropyl methyl cellulose phthalate; polymers derived from at least one acrylic acid, acrylic acid esters, methacrylic acid and methyacrylic acid esters such as methacrylic acid-methyl methacrylate polymer and theacrylic acid-ethylacrylate copolymers are also contemplated for use with the present invention. Additional polymers contemplated for use with the present invention include polymers derived from methylvinyl ether and maleic acid anhydride, polyvinylpyrrolidone, polyvinyl alcohols, and the like, as well as mixtures of any of the compounds named above.

Those of ordinary skill in the art would know at what concentrations to use these compounds. In one embodiment, polymer concentrations range from about 0.001% to about 5.0%. In another embodiment, the concentrations range from about 0.1 to about 1.0%. An example of sustained release formulation containing the corneal hardening agent glyceraldehyde would comprise glyceraldehyde at 3%, sodium carboxymethyl cellulose at 0.5% and bring the total volume to 100 milliliters.

In yet another embodiment of the present invention, corneal softening enzymes and compounds are administered to the cornea through use of a contact lens. As will be discussed in more detail below, the methods of the present invention involve the application of a rigid contact lens to a cornea in a suboptimal first conformation in order to reshape that cornea to a desired second conformation. In one embodiment of the present invention, the fitting of the contact lens and the administration of a corneal softening enzyme or compound occurs simultaneously. In an alternative embodiment of the present invention, the fitting of the contact lens and the administration of a corneal softening enzyme or compound occurs sequentially.

As an example of one embodiment of the present invention, a corneal softening amount of a corneal softening agent is loaded into a chamber inside a rigid contact lens, preferably one which is gas permeable. Alternatively, the enzyme or compound can be loaded or impregnated into a soft lens capable of taking up the enzyme or agent by soaking the soft lens in a solution containing the enzyme or agent. The enzyme or compound can also be loaded into a combination of a soft and a rigid lens.

In all of the following embodiments of a contact lens for administering a corneal softening enzyme or compound, the enzyme or compound is administered as it diffuses out of (is released from) the chamber in the lens or the material of the lens (if the enzyme or agent is soaked into a soft lens). Dosages for different refractive conditions and contact lens delivery vehicles can be optimised through routine experimentation by one of skill in the art.

In accordance with one method of administration through contact lenses of the present invention, corneal softening enzymes and compounds can be applied to the eye through the use of rigid contact lenses. These lenses can be made from known fluoro silicone acrylate lens materials, which are gas permeable. The lens is provided with an internal chamber for storing the corneal softening enzyme or compound. The chamber preferably comprises a radially symmetrical space encircling the entire lens between the anterior surface and posterior surface of the lens.

Rigid lenses for the present purpose can conveniently be made by lathe cutting, molding, or milling a posterior component and an anterior component from a contact lens button which, during fabrication, can be secured together to form a unitary lens using bonding techniques or adhesives known in the art. The chamber can be formed by lathe cutting an annular recess into the convex surface of the posterior component of the lens before the final lens fabrication. Any of a variety of dimensions can be used in accordance with the present invention, a preferred lens is provided with an annular chamber having a width of approximately 1.0 mm to about 1.5 mm and a depth of from about 0.05 mm to about 0.10 mm.

A plurality of microscopic holes are provided in the posterior portion of the lens to allow fluid communication between the chamber and the eye, thereby facilitating the timed release of the corneal hardening enzyme or agent into the cornea. These holes may be provided by mechanical or laser drilling, or by molding prior to assembling the anterior component and posterior component of the lens. In one embodiment the holes are drilled using a mechanical drill having a microcarbon drill bit.

The pumping action of the eyelids combined with natural tearing assists the release of the corneal hardening enzyme or agent through the tiny holes. Preferably, the holes are produced by mechanical drilling with a microcarbon bit and will have a diameter of from about 0.002 mm to about 0.010 mm, and preferably about 0.005 mm. The number and diameter of the holes can be varied to affect the time release characteristics, as will be apparent to one of skill in the art.

Day and/or night wear of these Enzyme Orthokeratology lenses may be used. The reshaping progress can be monitored using conventional methods.

The methods and compositions of the present invention can be used to correct myopia, astigmatism, and hyperopia.

In order that the invention may be readily understood and put into practical effect, particular preferred embodiments will now be described by way of the following non-limiting examples.

### EXAMPLES

### Example 1

Treatment of a Patient with Myopia Using MMP Manipulation

A patient with myopia has their corneal shape assessed and is fitted with an appropriate orthokeratology contact lens. For a week before commencement of wearing the contact lens, an MMP activator such as the commercially available Ciprofloxacin 0.3%, Ofloxacin 0.3%, Levofloxacin 0.5%, Gatifloxacin 0.3% or 0.5% Moxifloxacin.

The medication would be applied 4-6 times per day for one week. Alternatively, these medications can be given systemicvally eg. Levofloxacin at a dose of 30 mg/day for one week (which has been reported to alter human tendon structure; Kowatari et al., J Orthop Sci. 2004; 9(2): 186-90.).

The contact lens is then worn as in standard orthokeratology practice and the topical antibiotic continued. This has the added benefit of protecting against contact lens induced infection. Corneal shape is monitored through the treatment period by computerized corneal topography. When the preferred corneal shape has been achieved and is stable, the MMP-activating eyedrops are ceased.

Contact lens wear continues but MMP-inhibiting eyedrops are commenced.

The MMP inhibitor of choice is doxycyline as it is well tolerated, is known to penetrate the cornea well and has been extensively studied in corneal disease. Therapeutic doses can be achieved in the aqueous humor with 200 mg on day 1 then 100 mg bd (Zachariah S. Ann Ophthalmol. 1984 July; 16(7):672-4). In these doses it has been used to treat recurrent corneal erosion (Dursun et al. Am J Ophthalmol. 2001 July; 132(1):8-13.). It has also been used topically in doses of 0.025-0.1% eye drops that are commercially available (Leiter's Rx Compounding).

After 2 weeks of treatment, it is expected that the preferred corneal shape will have been achieved and both contact lens wear and medication can cease. It is expected (just as in refractive surgery) that some regression of the correction will occur and then retreatment would be initiated.

### Example 2

Alternative Ophthalmic MMP Activating Solution

Another method of corneal MMP activation is to use non-steroidal anti-inflammatory drugs (Reviglio et al. J Cataract Refract Surg. 2003 May; 29(5):989-97). Accordingly, other ophthalmic topical nonsteroidal antiinflammatory drug (NSAID) eyedrops include diclofenac sodium 0.1% (Falcon or Voltaren) and flurbiprofen sodium 0.03% (Ocufen). NSAIDs can also be given systemically and are known to have intraocular effects by this route—typical treatment regimes include: Naproxen (Naprosyn (250-500 mg bd) and Celebrex (100 mg bd).

An ideal combination for a composition that activates MMPs is thus an antibiotic such as a fluoroquinolone and an NSAID, in a formulation such as eyedrops to “soften” the cornea. This would allow the additional benefit of both antibiotic and anti-inflammatory effects. In addition since the mechanism of matrix metallproteinase activation is likely to be different, a synergistic effect could be expected and therefore a lower dose of each drug used.

Persons skilled in the art will appreciate that numerous variations and modifications will become apparent. All such variations and modifications which become apparent to persons skilled in the art, should be considered to fall within the spirit and scope that the invention broadly appearing before described.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

1. A method of moulding corneal tissue in an eye of a patient, the method comprising:
administering to the patient an effective amount of one or more compounds that activate at least one matrix metalloproteinase enzyme in the cornea and thereby soften the cornea;
providing a shaping means to the patient's cornea while said cornea is in a softened state until a desired corneal shape has been achieved;
ceasing the use of said shaping means.

2. The method according to claim 1, wherein the shaping means is a contact lens.

3. The method according to claim 1, wherein the one or more compounds that activate at least one matrix metalloproteinases enzyme in the cornea are selected from fluoroquinolone antibiotics, non-steroidal anti-inflammatory drugs (NSAIDS) and prostaglandin F(2alpha)(PGF (2alpha)) analogues.

4. The method according to claim 3, wherein the fluoroquinolone antibiotic is selected from ciprofloxacin, clinafloxacin, enoxacin, fleroxacin, gatifloxacin, gemifloxacin, grepafloxacin, levofloxacin, lomefloxacin, moxifloxacin, nalidixic acid, nomofloxacin, norfloxacin, ofloxacin, pefloxacin, sitafloxacin, sparfloxacin, temafloxacin and trovafloxacin, or any combination thereof.

5. The method according to claim 3, wherein the one or more compounds that activate at least one matrix metalloproteinase in the cornea are a fluoroquinolone antibiotic and a non-steroidal anti-inflammatory agent.

6. The method according to claim 1, wherein the desired corneal shape is retained by administration of an effective amount of one or more compounds that return the cornea to a hardened stated following administration of the one or more compounds that activate at least one matrix metalloproteinase enzyme in the cornea.

7. The method according to claim 6, wherein the one or more compounds that return the cornea to a hardened state are selected from inhibitors of one or more matrix metalloproteinases in the cornea, doxycycline, triptolide, cross-linking agents such as aldehydes, oxidative hardening agents such as copper sulfate and iron sulfate, enzymes such a lysyl oxidase and enzymes that hydroxylate collagen residues, enzymes that otherwise induce protein modifications that enhance corneal rigidity, corticosteroids, curcuminoids, galardin and medroxyprogesterone.

8. The method according to claim 7, wherein the matrix metalloproteinase inhibitor is selected from doxycycline, triptolide, corticosteroids and curcuminoids

9. The method according to claim 1, wherein the one or more compounds that activate one or more matrix metalloproteinases in the cornea are administered orally.

10. The method according to claim 1, wherein the one or more compounds that activate one or more matrix metalloproteinases in the cornea are administered topically.

11. The method according to claim 1, wherein the one or more compounds that activate one or more matrix metalloproteinases in the cornea are administered via a contact lens.

12. The method according to claim 1, wherein the one or more compounds that activate one or more matrix metalloproteinases in the cornea are administered intra-ocularly.

13. The method according to claim 1, wherein the one or more compounds that activate one or more matrix metalloproteinases in the cornea are administered parenterally.

14. The method according to claim 6, wherein the one or more compounds that return the cornea to a hardened state are administered orally.

15. The method according to claim 6, wherein the one or more compounds that return the cornea to a hardened state are administered topically.

16. The method according to claim 6, wherein the one or more compounds that return the cornea to a hardened state are administered via a contact lens.

17. The method according to claim 6, wherein the one or more compounds that return the cornea to a hardened state are administered intra-ocularly.

18. The method according to claim 6, wherein the one or more compounds that return the cornea to a hardened state are administered parenterally.

19. The method according to claim 1, wherein the moulding of the cornea is part of an orthokeratology procedure.

20. The method according to claim 1, wherein the moulding of the cornea is performed to enhance an outcome of a LASIK procedure.

21. The method according to claim 1, wherein the moulding of the cornea is performed to enhance an outcome of a LASEK or PRK procedure.

22. The method according to claim 1, wherein the moulding of the cornea is performed following cataract surgery, wherein a replacement lens that is used in said cataract surgery that does not provide sufficient visual acuity.

23. A composition for softening corneal tissue in a patient, said composition comprising an effective amount of one or more compounds that activate at least one matrix metalloproteinase enzyme in the cornea, optionally in association with one or more pharmaceutically acceptable carriers or excipients.

24. The composition according to claim 23, wherein the one or more compounds that activate at least one matrix metalloproteinases enzyme in the cornea are selected from fluoroquinolone antibiotics, non-steroidal anti-inflammatory drugs (NSAIDS) and prostaglandin F(2alpha)(PGF (2alpha)) analogues.

25. The composition according to claim 24, wherein the fluoroquinolone antibiotic is selected from ciprofloxacin, clinafloxacin, enoxacin, fleroxacin, gatifloxacin, gemifloxacin, grepafloxacin, levofloxacin, lomefloxacin, moxifloxacin, nalidixic acid, nomofloxacin, norfloxacin, ofloxacin, pefloxacin, sitafloxacin, sparfloxacin, temafloxacin and trovafloxacin, or any combination thereof.

26. The composition according to claim 24, wherein the one or more compounds that activate at least one matrix metalloproteinase are a fluoroquinolone antibiotic and a non-steroidal anti-inflammatory agent.

27. A composition for hardening corneal tissue in a patient following corneal softening by activation of one or more matrix metalloproteinases, said composition comprising one or more corneal-hardening agents, optionally in association with one or more pharmaceutically acceptable carriers or excipients.

28. The composition according to claim 27, wherein the one or more corneal-hardening agents are selected from an inhibitor of one or more matrix metalloproteinases in the cornea, doxycycline, triptolide, cross-linking agents such as aldehydes, oxidative hardening agents such as copper sulfate and iron sulfate, enzymes such a lysyl oxidase and enzymes that hydroxylate collagen residues, enzymes that otherwise induce protein modifications that enhance corneal rigidity, corticosteroids, curcuminoids, galardin and medroxyprogesterone.

29. The composition according to claim 28, wherein the matrix metalloproteinase inhibitor is selected from doxycycline, triptolide, corticosteroids and curcuminoids
